(19) United States
(12) Patent Application Publication
(10) Pub. No.: US 2016/0220582 A1
(43) Pub. Date: Aug. 4, 2016

(54) COMBINATION OF LUPEOL ACETATE AND CURCUMIN USED FOR THE TREATMENT OR PREVENTION OF ACTIVATED OSTEOCLAST PRECURSOR ASSOCIATED DISORDERS

(71) Applicant: NATIONAL YANG-MING UNIVERSITY, Taipei City (TW)

(72) Inventors: Jeng-Jong HWANG, Taipei City (TW); Wei-Hsun WANG, Taipei City (TW)

(21) Appl. No.: 14/851,461

(22) Filed: Sep. 11, 2015

(30) Foreign Application Priority Data
Jan. 29, 2015 (TW) 104103096

(51) Int. Cl. A61K 31/56; A61K 31/12

(52) U.S. Cl. CPC A61K 31/56; A61K 31/12

(57) ABSTRACT

The present invention relates to synergistic combinations of lupeol acetate and curcumin at low dosage, and their use for the treatment or prevention of activated osteoclast precursor related diseases, including rheumatoid arthritis and osteoporosis.

### BACKGROUND OF THE INVENTION

1. Technical Field of the Invention

The present invention relates to a synergistic combination of lupeol acetate (LA) with curcumin and its use in the treatment or prevention of activated osteoclast precursor associated disorders. Especially, the present invention relates to a composition of lupeol acetate and curcumin at low dosage, used for regulating immune functions and inhibiting rheumatoid arthritis (RA) or osteoclastogenesis-related diseases.

2. Background

Reumatoid arthritis (RA) is a chronic autoimmune disorder that is closely correlated with the excessive activation of macrophages. The activation of macrophages in the joint will release proinflammatory cytokines, and attract more immune cells to infiltrate, result in more severe inflammatory response, and causes the disintegration of articular cartilage and bone injuries, which can lead to joint deformity at late stage and substantial loss of function and mobility. Macrophages may further differentiate into osteoclasts and lead to bone erosion in the joint cavity, which is the main reason for the progression of RA.

The major pharmaceuticals used in clinical treatment of RA are steroids, non-steroid anti-inflammation drugs, and certain biological agents against cytokine, such as TNF-α blockers, anti-IL-1β, anti-IL-6 antibodies, and the like (Breedveld F C. Arthritis Res 2002, 4(2):27). Such therapeutic agents are not only expensive, but also possess certain degree of side effects.

Lupeol acetate (LA), a type of triterpene, is an ingredient in the extraction of Shea nut, and exists in the mango, cabbage and green pepper. Lupeol acetate has a chemical structure similar to sterols, and has been known with capability of anti-inflammation, anti-oxidation, anticancer and immunomodulation (see, Akihisa T et al., J Oleo Sci 2010, 59(6):273-280; Saleem M. Cancer Lett 2009, 285(2):109-115; Siddique H R, Saleem M. Life Sci 2011, 88(7-8):285-293). It is also demonstrated that LA can effectively mitigate the inflammatory condition induced by carrageenan in mice (Lucetti D L et al. J Inflamm 2010, 7(60)).

In addition, US Patent Application no. 20120177754 has disclosed extraction of lupeol acetate from Boswellia frereana, and the significant therapeutic effect of lupeol acetate in inhibiting inflammation and treatment of rheumatoid arthritis. However, the animal model experiments show that long-term use of high doses (100 mg/kg, 12 days) is necessary for rheumatoid arthritis treatment in mice even a highly pure extract of natural lupeol acetate (95%) is used in the therapy.

Curcumin is a principal curcuminoid extracted from Curcuma Tonga (also known as Turmeric), which is a member of the ginger family (Zingiberaceae), and has been used in curry powder as a common and cheap spice component. Curcumin has been reported with effects of immune modulation and anticancer, and has been shown to have antioxidant, anti-inflammatory and anti-atherosclerosis effects in several animal experiments. It is also known to inhibit the occurrence of inflammation and progression of arthritis in mice. However, the clinical application of curcumin is limited by its poor bioavailability.

Therefore, the present invention contemplates to combine lupeol acetate with curcumin for significantly reducing the cost of drug production, and achieving synergistic effects in the treatment or prevention of activated osteoclast precursor associated disorders to benefit more kinds of patients in clinical use.

### SUMMARY OF INVENTION

Based on the purpose described above, the present invention finds that the combination of lupeol acetate (LA) with curcumin at low doses significantly reduced the activation of macrophages and osteoclastogenesis. The composition of lupeol acetate and curcumin will not only create synergistic effects for inhibiting inflammation and alleviating bone loss at a reduced dosage of lupeol acetate, but also improve the bioavailability of curcumin for clinical application.

Accordingly, in one aspect, the present invention relates to a pharmaceutical composition for treating or preventing osteoclastogenesis associated disorders, comprising lupeol acetate and curcumin combined at certain content or proportion of the composition.

In one preferable embodiment of the present invention, the composition comprises 25-50 mg/kg of lupeol acetate and 40-50 mg/kg of curcumin, and pharmaceutically acceptable carrier, diluent or excipient. In some embodiments of the present invention, the lupeol acetate and the curcumin are combined at a ratio of 0.5:1 to 1:2, and preferably at a ratio of 1:1 to 1:2.

In certain embodiments of present invention, the osteoclastogenesis associated condition includes rheumatoid arthritis (RA). In other embodiments of present invention, the osteoclastogenesis associated condition includes osteoporosis. In a further embodiment of present invention, the osteoporosis is a sterol anti-inflammatory agent triggered osteoporosis.

In another aspect, the present invention relates to a method treating or preventing an osteoclastogenesis associated condition by administering the pharmaceutical composition comprising lupeol acetate and curcumin, combined at a ratio of 0.5:1 to 1:2.

In certain embodiments, the osteoclastogenesis is suppressed by inhibiting a differentiation of osteoclast from macrophage.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic diagram of the mechanism of the combined action of lupeol acetate and curcumin in the treatment of rheumatoid arthritis and osteoporosis. In FIG. 1, the upward arrow indicates an enhancing effect and the downward arrow indicates an inhibitory effect.

FIGS. 2A-2C show the cell viability of RAW 264.7 mouse macrophage cell line after the drug treatment of lupeol acetate (LA), curcumin (Cur) and combination evaluated with MTT assay. In FIG. 2A, RAW264.7 cells were treated with different concentrations of lupeol acetate (10, 20, 40, 80 μM); in FIG. 2B, RAW 264.7 cells were treated with curcumin at concentration of 2.5, 5, 7.5 or 10 μM); and in FIG. 2C, RAW264.7 cells were treated with a combination of lupeol acetate and curcumin (10 μM Cur+10 μM LA, 10 μM Cur+20 μM LA, 10 μM Cur+40 μM LA or 10 μM Cur+80 μM LA) for 24 h. Cell viability was analyzed by MTT (3-(4,5-dimethylthiazol-2-yl)-2,5-diphenyl tetrazolium bromide). The experimental results are compared to the respective control groups, which resultant value is set to 1.

FIGS. 3A-3E show the golden section determined from the effects of combination treatment on LPS-stimulated macrophage. In FIG. 3A, significant decrease of pro-inflammatory TNF-α release in the group of 40 μM LA+10 μM Cur was found after the treatment of various combinations of LA+Cur on LPS-stimulated RAW 264.7 mouse macrophage cells. ^{aaa}: p<0.001 as compared with 20 μM LA alone; ^{bbb}: p<0.001 as compared with 40 μM LA alone, ^{ccc}: p<0.001 as compared with 10 μM Cur alone. FIG. 3B and FIG. 3C show the significant decrease of other two pro-inflammatory cytokines, IL-6 and IL-1β after the combination treatment (40 μM LA+10 μM Cur), respectively. FIG. 3D and FIG. 3E show that the mean fluorescence intensity of cellular antigens CD80 and CD86 were markedly decreased. ^{###}: p<0.001 as compared with combination group; ***: p<0.001 and **: p<0.01 as compared with untreated group.

FIGS. 4A-4B show that the ability of cell migration is diminished by the combination of lupeol acetate and curcumin via down-regulation of migrated-related proteins. In FIG. 4A, LA, Cur and combination (40 μM LA+10 μM Cur) were added to the upper chamber an hour prior to the addition of 1 μg/ml LPS to the lower chamber. After plates were incubated for 24 h, the ability of cell migration was observed and counted to quantification under microscope. FIG. 4B shows that the expression of migration-related proteins like COX-2 and CCL-2 were significantly reduced, and the quantification was shown in lower panels.

FIGS. 5A-5B show the inhibitory effects of lupeol acetate combined with curcumin on RANKL-induced osteoclastogenesis by regulating NF-κB. FIG. 5A shows that receptor activation of nuclear factor κB ligand (RANKL) could induce differentiation of macrophage into osteoclasts. Combination treatment (LA+Cur) was shown to inhibit RANKL-induced osteoclast formation. FIG. 5B shows that combination treatment regulates the osteoclastogenesis-related transcription factor, NFATc1, as determined by RT-PCR. (**: p<0.01 and ***: p<0.001 as compared with RANKL group. ##: p<0.01 and ###: p<0.001 as compared with combination group).

FIGS. 6A-6F show the evaluation of therapeutic efficacy of LA alone (25 mg/kg and 50 mg/kg, respectively), LA+Curcumin (25 mg/kg+50 mg/kg), and 50 mg/kg Curcumin treatments on day 32 after first immunization in the CIA-induced RA animal model. FIG. 6A shows the arthritis onset of clinical symptoms in combination (LA+Cur) treated mice were significantly delayed as compared with non-treated CIA mice. FIGS. 6B and 6D show the arthritis scores in each group (the highest score is 16 points for each mouse), indicating the severity of clinical score is significantly decreased by combination treatment. In the figures, arrow indicates the swelling of inflammation site of toe and ankle. FIGS. 6C and 6E show the incidence of CIA in each group, which is significantly delayed in the combination group (LA 25 mg/kg+curcumin 50 mg/kg). FIG. 6F shows the changes in body weight of mice after giving the drugs (alone and in combination), indicating no toxic effects on the treated animal.

FIG. 7A shows the uptake of ¹⁸F-labelled fluorodeoxyglucose (¹⁸F-FDG) in joints of DBA/1J mice with collagen-induced arthritis. FIG. 7B shows the bone density of femur in mice tracked by micro-CT imaging. The bone density in LA and curcumin treated mice were significantly higher than that of CIA mice. (n=9) *: p<0.05, **: p<0.01, ***: p<0.001 as compared with that of CIA group for each scan time.

FIGS. 8A-8E show the combination treatment inhibit the expression of arthritis-related proteins and promote the immunosuppressor factors in CIA mice. In FIG. 8A, mice were sacrificed at the peak of arthritis (on day 32), the proteins were extracted from legs of each group. The protein levels of angiogenesis, cell migration, bone erosion and immunosuppressor factor were assayed with ex vivo Western blotting. LA25 (lupeol acetate 25 mg/kg), LA50 (lupeol acetate 50 mg/kg), and LA25+Cur50 (lupeol acetate 25 mg/kg+curcumin 50 mg/kg) show the improvement of arthritis-related proteins, especially the combination group. FIG. 8B shows the suppression of NF-κB activation with ex vivo electrophoretic mobility shift assay (EMSA). The activation of NF-κB is significantly suppressed in LA groups (LA25 and LA50) and (LA25+Cur50) group, especially the combination group shows the maximum suppression. FIGS. 8C-8E show the effects of LA, Cur and combination treatment on serum cytokines in CIA mice. The expressions of IL-17 and TNF-α (FIG. 8C), IL-6 (FIG. 8D) and BAFF (FIG. 8E) reached the peak on day 32, and were significantly decreased in the combination groups (n=9). *: p<0.05, **: p<0.01, ***: p<0.001 as compared with CIA alone. #: p<0.05, ##: p<0.01, ###: p<0.001 as compared with the combination group.

FIGS. 9A-9B show the increased expression of Treg in each groups. Mice were sacrificed on day 32 after first immunization of animal experiment, and the spleen (FIG. 9A) and inguinal lymph nodes (FIG. 9B) were removed for the Treg analysis, respectively.

FIGS. 10A-10C show the therapeutic effect on LA alone and combination (25 mg/kg LA plus 50 mg/kg curcumin) on CIA mice, and using Celecoxb group (10 mg/kg, a Cox-2 inhibitor) for comparison. FIG. 10A shows the protein levels of angiogenesis, cell migration, bone erosion and immunosuppressor factors in each groups examined by ex vivo Western blot. FIG. 10B shows the serum level of IL-6 in each group on day 21, 32 and 43. (*p<0.05, **p<0.01, ***p<0.001 as compared with that of CIA group; ^{#}p<0.05, ^{##}p<0.01, ^{###}p<0.001 comparison between treated groups). FIG. 10C shows the histopathology of ankle joints in CIA mice treated with LA alone, combination (LA+Cur) and Celecoxb, respectively. Histopathology examination of joints was evaluated by H & E stain (original magnification 100×).

### DETAILED DESCRIPTION OF THE INVENTION

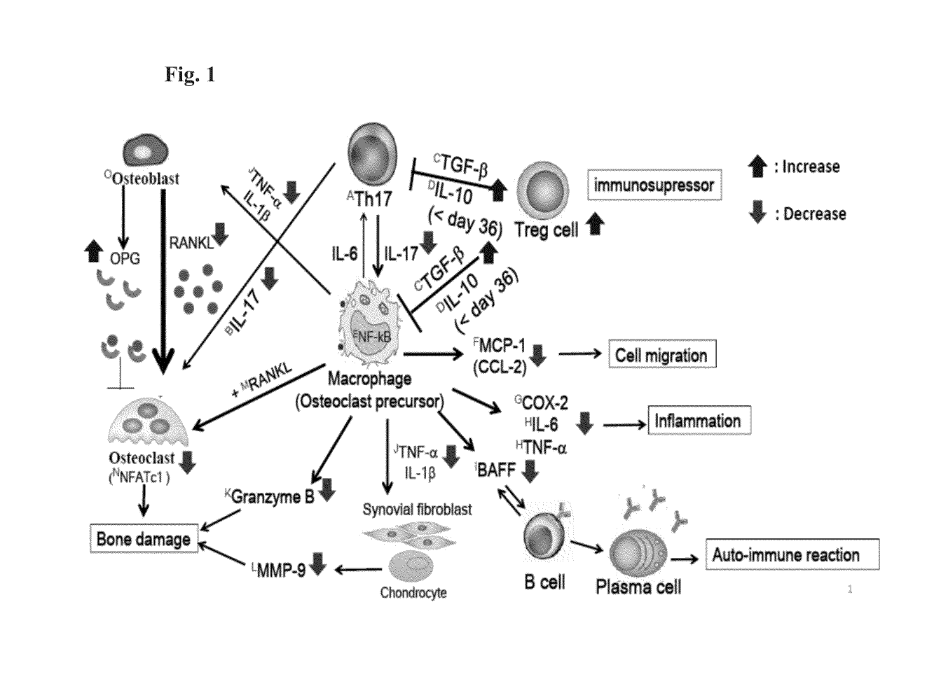

As used herein, term “activated osteoclast precursor associated disorders” refers to the disorders caused by the over-activation of osteoclast precursor. The “osteoclast precursor” is refers to macrophage in general. As shown in FIG. 1, the over-activation of macrophage triggers a series of immune response, inducing the production and secretion of proinflammatory cytokines (TNF-α, IL-1β, IL-6, IL-17) and cell migration related proteins (COX-2 and MCP-1), which could cause a series of inflammation responses and autoimmune diseases such as rheumatoid arthritis (RA).

Once macrophage is activated, it would secret TNF-α, IL-1β, and cause osteoblast to reduce the secretion of OPG and increase the secretion of RANKL (receptor activator of nuclear factor kappa-B ligand). The binding of OPG and RANKL can reduce the formation of osteoclasts. Macrophage acts as a precursor in osteoclast. There are RANKL receptors on its cell membrane, once binding with RANKL would trigger osteoclastogenesis to differentiate into osteoclasts, and further result in bone erosion and osteoporosis. Thus, in certain embodiments of the present invention, the activated osteoclast precursor associated disorders include rheumatoid arthritis and osteoporosis.

The other characteristics and advantages of the present invention will be further illustrated and described in the following examples. The examples described herein are using for illustrations, not for limitations of the invention.

### Example 1

### The Cytotoxicity of Lupeol Acetate (LA), Curcumin (Cur) and Combination on RAW 264.7 Cell Line

RAW 264.7 cells (a mouse macrophage cell line) were treated with different concentrations of lupeol acetate (10, 20, 40, 80 μM), curcumin (2.5, 5, 7.5, 10 μM) and combination (10 μM Cur+10 μM LA, 10 μM Cur+20 μM LA, 10 μM Cur+40 μM LA, 10 μM Cur+80 μM LA) for 24 h. Cell viability was evaluated with MTT assay, and compared to the results of the control group.

5×10⁴ RAW264.7 cells/well were seeded in 96-well culture dishes, and treated with directed concentration of lupeol acetate (LA), curcumin (Cur) and combination for 24 hours after the 24-hr attachment. After removing the culture medium, 100 μl of 0.5 mg/ml MTT solution was added, and the cells were incubated at 37° C. for 4 hours. The mitochondrial enzyme (succinate dehydrogenase, SDH) in living cells will react with tetrazolium bromide in the MTT solution and form formazan blue-purple crystals. The MTT solution was removed and 200 μl DMSO was added to dissolve the blue-purple crystal, and then the O.D. (optical density) value was read under wavelength of 570 nm with an ELISA reader (TECAN Sunrise, USA). The relative cell viability is calculated by comparing the absorbance value of drug treated group with that of the control group (setting the value of control group as 100%).

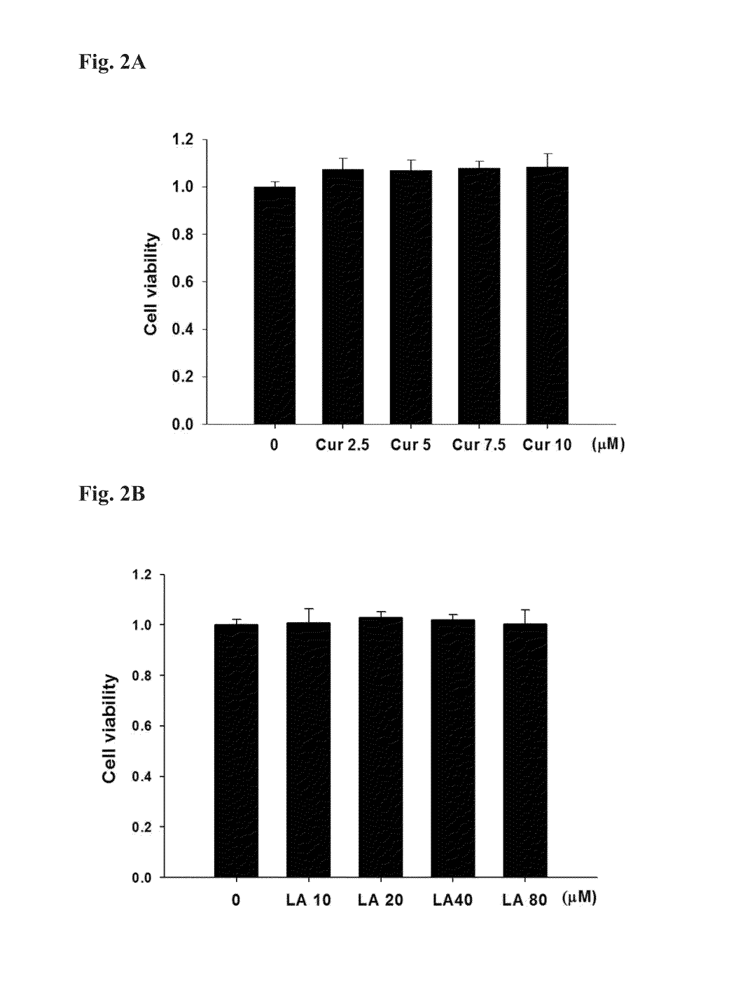

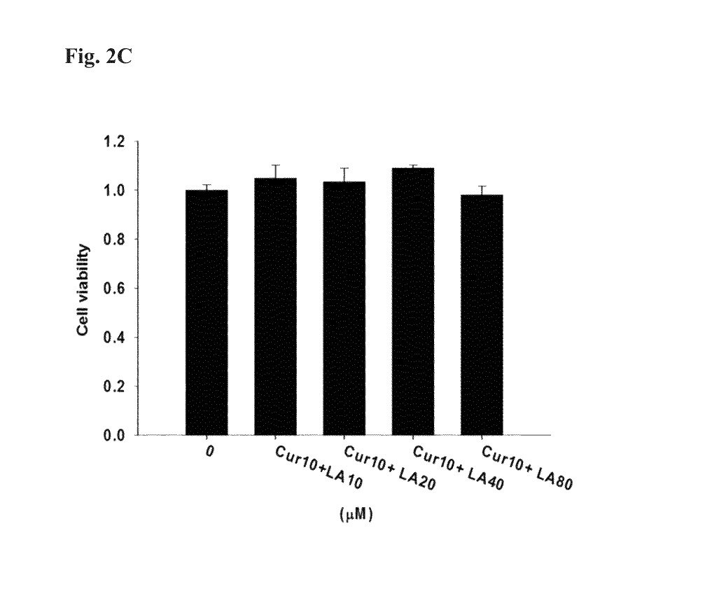

From the analytic results shown in FIG. 2, lupeol acetate (LA), curcumin (Cur) and combination of LA with Cur all have no cytotoxic effects on RAW264.7 cells under indicated concentrations.

### Example 2

### Suppression of the Inflammatory Mediator Production by Combination Treatment of Lupeol Acetate with Curcumin

In this example, the expressions of TNF-α, IL-6 and IL-1β, all are pro-inflammatory cytokines, assayed by ELISA are used to determined the optimal combination of LA and curcumin. RAW264.7 macrophages were pretreated with different concentrations of LA, curcumin and combination for an hour prior to the addition of 1 μg/ml LPS, then incubated for another 24 h at 37° C., the supernatants from each group were collected, and the expression levels of TNF-α, IL-1β and IL-6 were detected by ELISA.

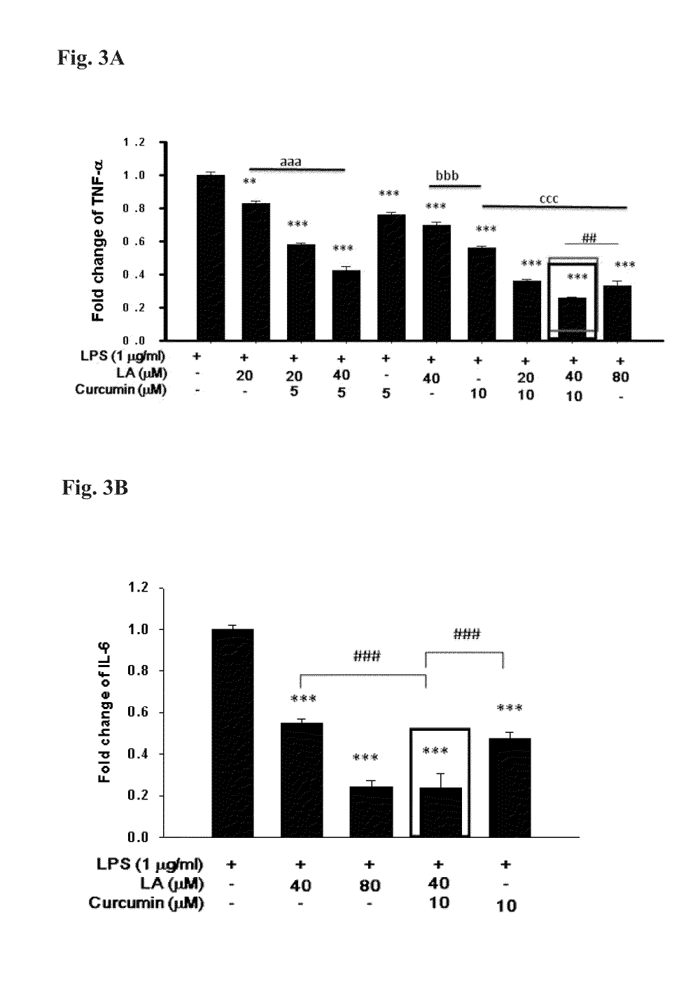

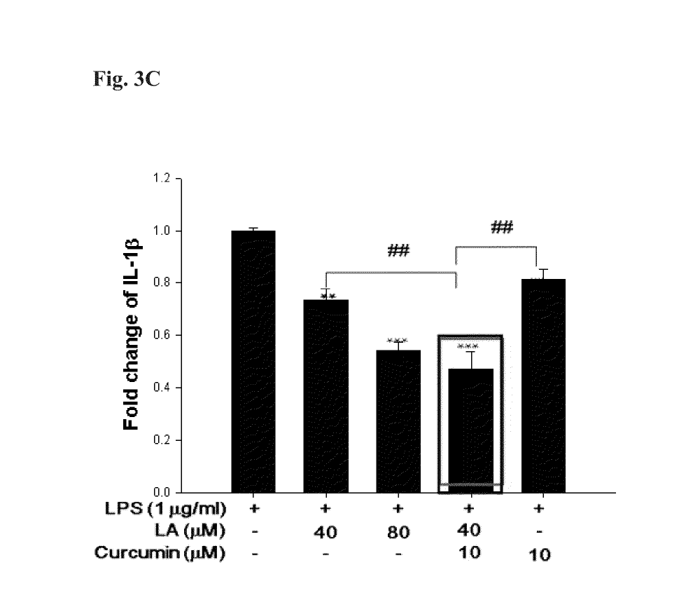

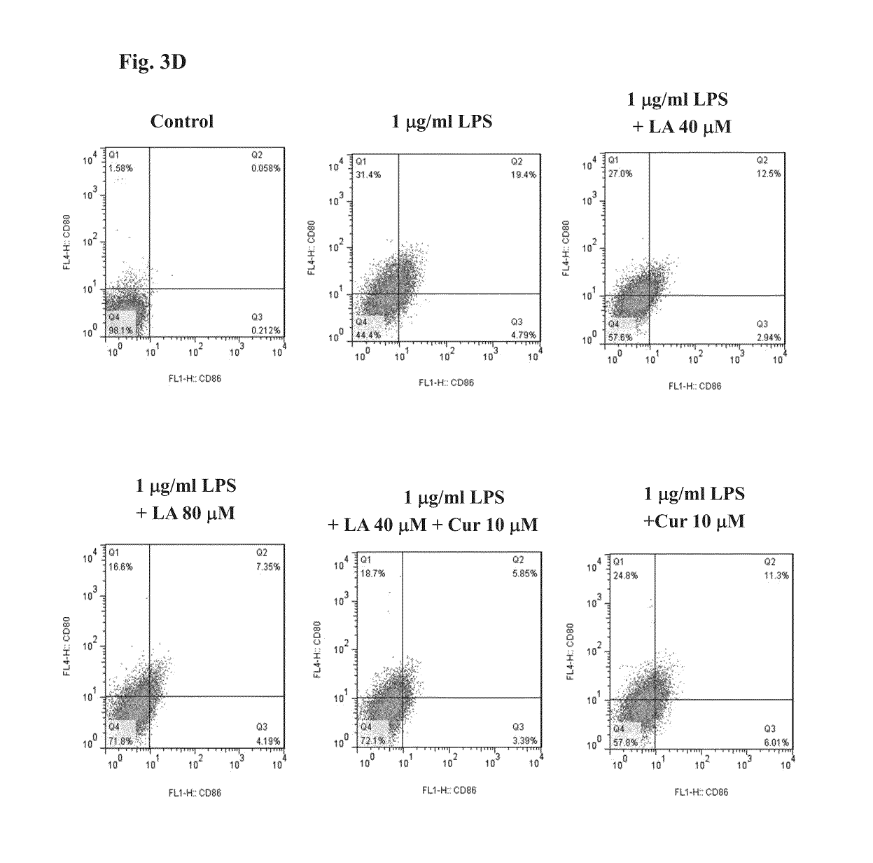

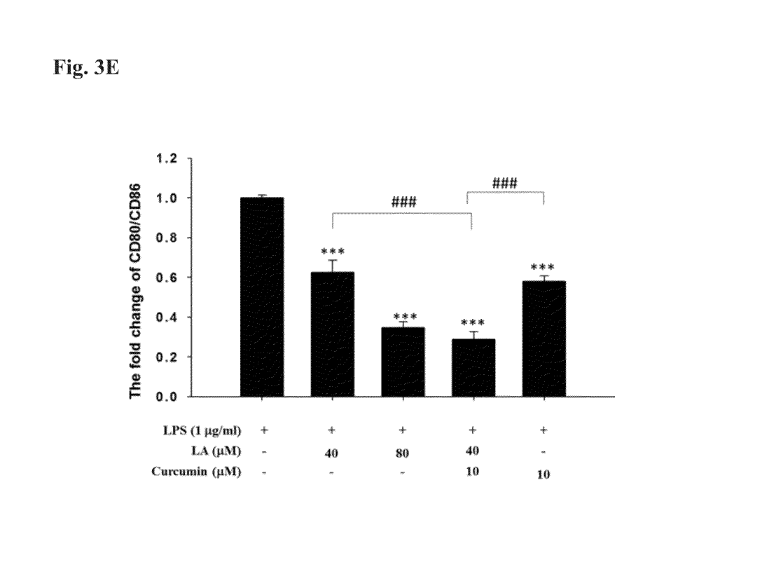

As shown in FIG. 3A, after treated with various combinations of LA+Cur on LPS-stimulated RAW264.7 mouse macrophage cells, significant decrease of pro-inflammatory TNF-α release was found in the group of 40 μM LA+10 μM Cur, with the same or slight better inhibitory effect compared to that of 80 μM LA. Combination index (CI) analysis demonstrates that 40 μM LA combined with 10 μM Cur shows the synergistic effect. [synergism (CI<1), additive effect (CI=1), and antagonism (CI>1)]. Both IL-6 and IL-1β also showed the similar results (see FIGS. 3B and 3C). Therefore, the combination (40 μM LA+10 μM Cur) was used for further studies.

In addition, under LPS stimulation, CD86 and CD80, the two co-stimulation factors expressed on the surface of macrophage, were decreased after the combination treatment of 40 μM LA+10 μM Cur (FIGS. 3D and 3E), indicating that the combination treatment can inhibit the formation of co-stimulator CD80⁺CD86⁺ during macrophage activation.

### Example 3

### Combination Treatment Could Inhibit LPS-Mediated Migration Ability and Expression of Inflammation Related Proteins in RAW 264.7 Cells

Macrophages are usually immobile but become actively mobile when stimulated by inflammation, immune cytokines and microbial products. To examine whether LA+Cur could decrease the migratory ability of macrophage, LPS was used to activate RAW264.7 macrophage cells. Transwell assay was used to determine the migration ability of macrophage cells which could be changed by combination treatment.

1×10⁵ RAW264.7 macrophages cells were seeded in Transwell upper chamber (5.0 μm polycarbonate membrane, 6.5 mm insert, 24-well plate, Corning, USA). LA, Cur and combination (40 μM LA+10 μM Cur) are added into the upper chamber an hour prior to the addition of 1 μg/ml LPS to the lower chamber. After incubating for 24 hr at 37° C., 5% CO₂, the medium was removed from the Transwell upper chamber and the membrane was washed twice with PBS. The migrated cells on the membrane were fixed with the fixative (methanol:glacial acetic acid=3:1) for 15 min and stained with hematoxylin for 10 min. Hematoxylin positive cells in each group were counted for five views under the microscope (100×) and were quantified.

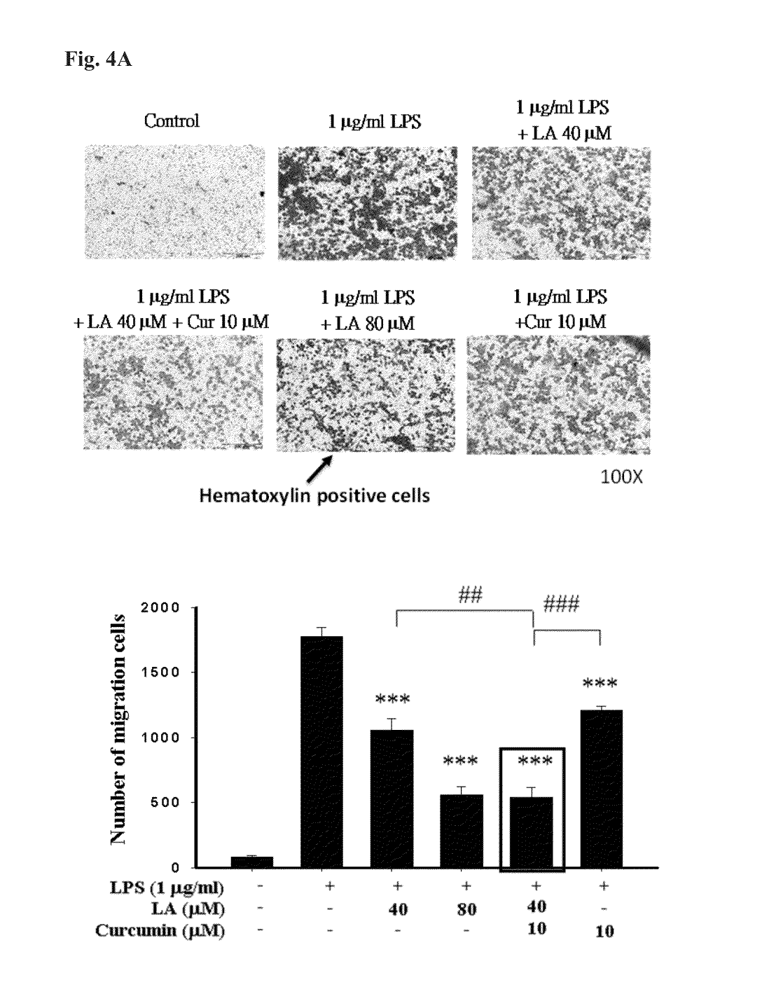

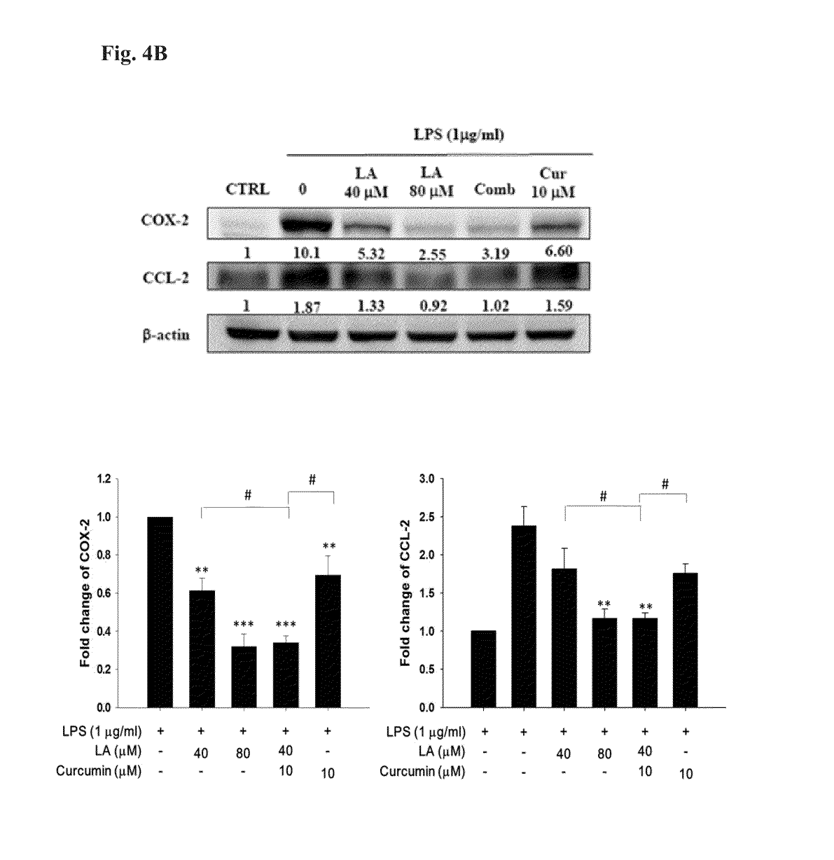

As shown in FIG. 4A, untreated cells (control) exhibit significant movement under the attraction of 1 μg/ml LPS stimulation. However, the macrophage migration have been significantly reduced by the treatment of combination (40 μM LA+10 μM Cur).

The protein levels of COX-2 and CCL-2 were found to modulate cell migration under LPS stimulation. Therefore, the expressions of COX-2 and MCP-1 were further confirmed by Western blotting. Results have shown that LPS stimulation would cause large amount of cells to migrate. However, cells harvested from LPS-stimulated RAW264.7 cells treated with 40 μM LA+10 μM Cur, and the protein levels were assayed with Western blotting. As shown in FIG. 4B, the expressions of migration-related proteins such as MCP-1 and COX-2 were significantly reduced. In summary, these results show that combination treatment of 40 μM lupeol acetate and 10 μM curcumin reduces the migration ability significantly via down-regulation of COX-2 and CCL-2 expressions in macrophage, and with the same effect as that of 80 μM LA alone.

### Example 4

### Inhibitory Effects of Combination of Lupeol Acetate and Curcumin on the Formation of Osteoclasts Induced by RANKL

The generation and metabolism of bones are kept in a state of dynamic equilibrium, and the destruction of this equilibrium will cause the damage of bones. In rheumatoid arthritis, there is excessive osteoclast formation occurred and resulting in the erosion of bone.

To evaluate the effects of the combination of present invention on the suppression of rheumatoid arthritis, we firstly analyze the differentiation of osteoclasts from certain cells using the tartrate-resistant acid phosphatase (TRAP) staining method. The TRAP staining is used to detect the internal acid phosphatase activity of leukocytes in a blood, bone or tissue sample. Because osteoclasts contain acid phosphatase, we can use this staining method to determine the formation of osteoclast cells.

1×10⁴ RAW 264.7 cells were seeded in 96-well plates and treated with LA (40 μM LA, 80 μM LA), Cur (10 μM curcumin) and combination (40 μM LA+10 μM curcumin) co-incubated with 100 nM RANKL in α-MEM medium supplemented with 10% bovine calf serum (BCS, Sigma, USA), 1% L-glutamine (Gibco-BRL, CA, USA) and 1% penicillin-streptomycin (Gibco-BRL, CA, USA). On Day 5, cells were stained for tartrate-resistant acid phosphatase (TRAP). After removing the supernatant of culture, the cells are rinsed twice with PBS, and then fixed with 3.7% paraformaldehyde for 1 hour. The fixed cells are washed with PBS. The Acid Phophatase Leukocyte kit (TRAP stain, Cat. 387-A, Sigma-Aldrich, USA) is used in the TRAP staining and the method is briefly described as follows. Before staining, the temperature of ddH₂O used for adjuvant preparation is confirmed to be 37° C. The Fast Garnet GBC base and sodium nitrite solution at equal volumes are uniformly mixed for 30 seconds and incubated at room temperature for at least two minutes.

Then following the procedures described in the instruction manual, a staining agent is prepared by adding a well-mixed solution of 1 ml Fast Garent GBC base, 0.5 ml Naphthol AS-BI phosphate solution, 2 ml acetate solution and 1 ml tartrate solution to 45 ml of 37° C. ddH₂O. The staining agent is uniformly mixed and added to each well of 96-well plate at 100 μl aliquot, and then placed in a 37° C. dark incubator for one hour. After the reaction, the 96-well plate is wetted by ddH₂O, and stained with a hematoxylin solution included in the kit for ten minutes, then rinsed with tap water and air dried. Finally, the osteoclast differentiation is observed under a microscope, those contain 3 or more nuclei will be identified as osteoclast.

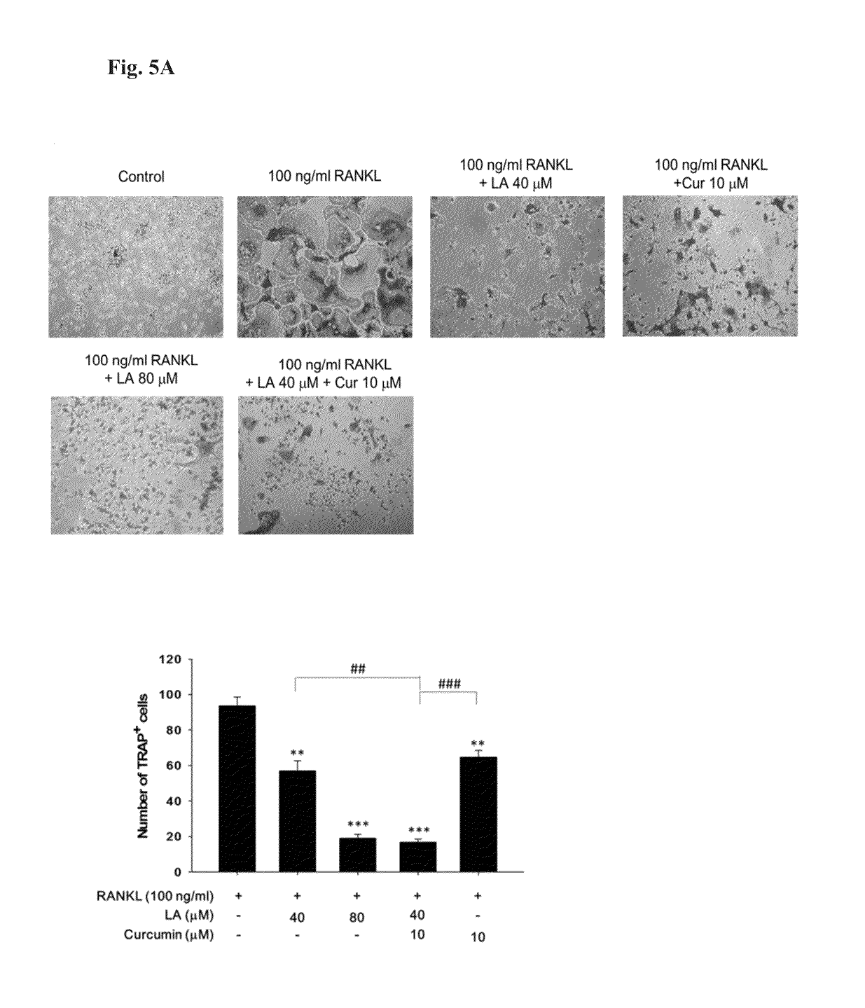

As shown in FIG. 5A, macrophages differentiate into osteoclasts under the stimulation of receptor of activation of NF-κB ligand (RANKL), and a decrease in the differentiation of macrophages into osteoclasts is indeed observed after administration of drugs. Results from the above experiments have shown that the combination treatment can inhibit the formation, maturation, and differentiation of the osteoclast by modulating the activity of NF-κB.

Next, the real time Q-PCR is used for analyzing the change of NFATc1 (nuclear factor of activated T cell, the major factor of osteoclast proliferation and known as cytoplasmic 1) under the RANKL induction and drug treatments. As shown in FIG. 5B, the expression level of transcriptional factor NFATc1 is increased by the stimulation of RANKL, and is significantly reduced after the treatment of drugs.

### Example 5

### Clinical Symptoms of Arthritis were Significantly Reduced by Combination Treatment in Collagen-Induced Arthritis (CIA) Mice

The animal model used in present studies of rheumatoid arthritis is the collagen-induced arthritis animal model; the progression of rheumatoid arthritis in this animal model is similar to that in human. In this embodiment, bovine type II collagen combined with complete Freund's adjuvant (CFA) is used to induce rheumatoid arthritis in DBA/1J mice, also known as collagen-induced arthritis (CIA) mice.

The type II collagen is a major component of cartilage, and the use of heterologous (bovine) collagen will induce the production of anti-CII antibody in mice, resulting in the self-immune response to attack its own joint cartilage. In the early stage, the complement system is initially activated to attract neutrophils and macrophages, and stimulate the release of inflammatory cytokines from the activated cells. The inflammatory mediators will further affect T cells, B cells and macrophages to produce a more severe inflammation and further attack joints to progress into rheumatoid arthritis.

Eight-week-old DBA/1J mice (purchased from Jackson lab, ME, USA and housed in the Animal center of National Yang Ming University under pathogen-free conditions according to the Institutional Animal Care and Committee guidelines) were used. 100 μl of arthritis-inducing adjuvant (prepared by mixing Complete Freund's Adjuvant (5 mg/ml heat-killed M. tuberculosis in incomplete Freund's Adjuvant) (Chondrex, WA, USA) with equal volume of bovine type II collagen (2 mg/ml solution in 0.05 M acetic acid) (Chondrex, WA, USA)) is injected into the dermis of tail (intra-dermal, i.d.) using a 30 G syringe; and a 50 μl second dose of same ingredients is injected in the same way at an interval of 21 days. The symptoms are produced at about six days after the second dose injection, with an induction rate of 100%. The CIA mice were established and treated with LA 25 mg/kg alone, LA 50 mg/kg alone, Cur 50 mg/kg alone and LA 25 mg/kg+Cur 50 mg/kg after second immunization once per day. Using animals treated with deionized distilled water (ddH₂O) containing 0.1% dimethyl sulfoxide (DMSO, Sigma, USA) as the normal mice group. Signs of arthritis were monitored until Day 43.

Clinical score is used to assess the redness and swelling of limbs of each mouse. Arthritis score: 0=normal, 1=slight swelling and/or erythema, 2=extensive swelling and/or erythema, 3=joint distortion and/or rigidity, 4=very inflamed and swollen paw or ankylosed paw. Each limb will be measured, the scores of four limbs will be summed up, and the maximum score of each mouse will be 16.

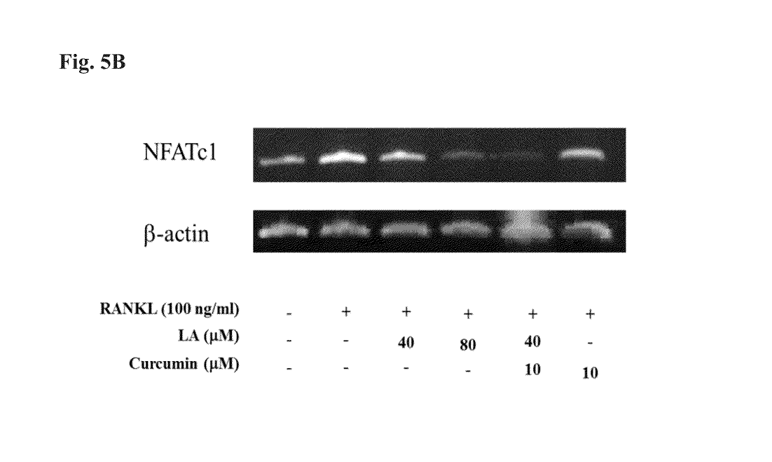

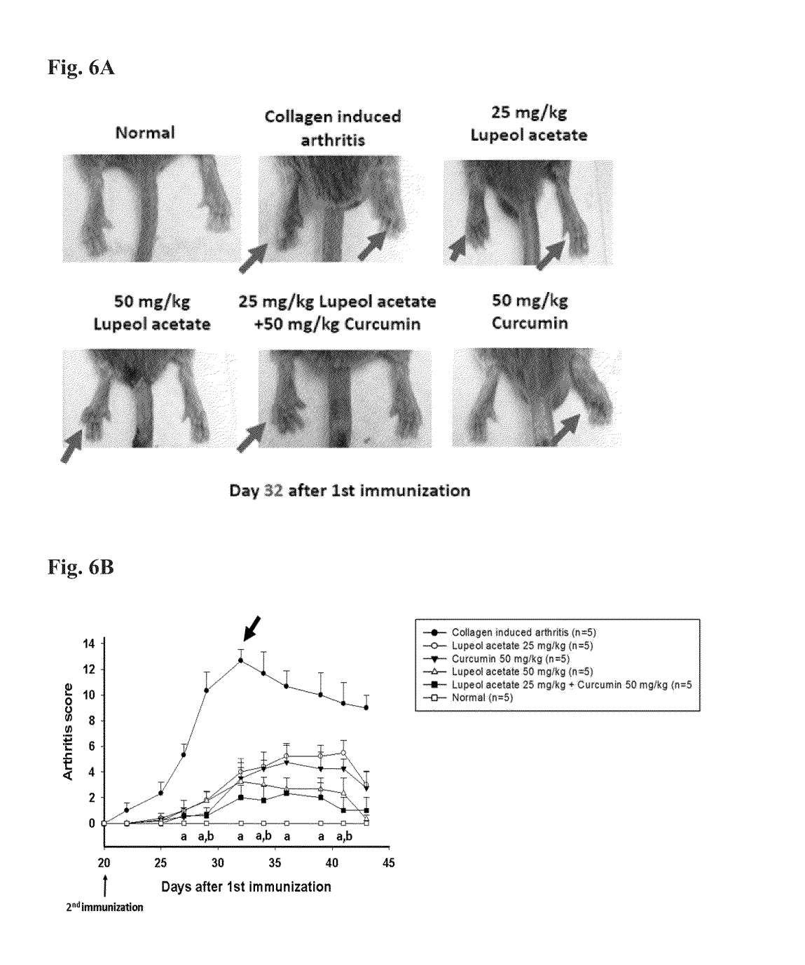

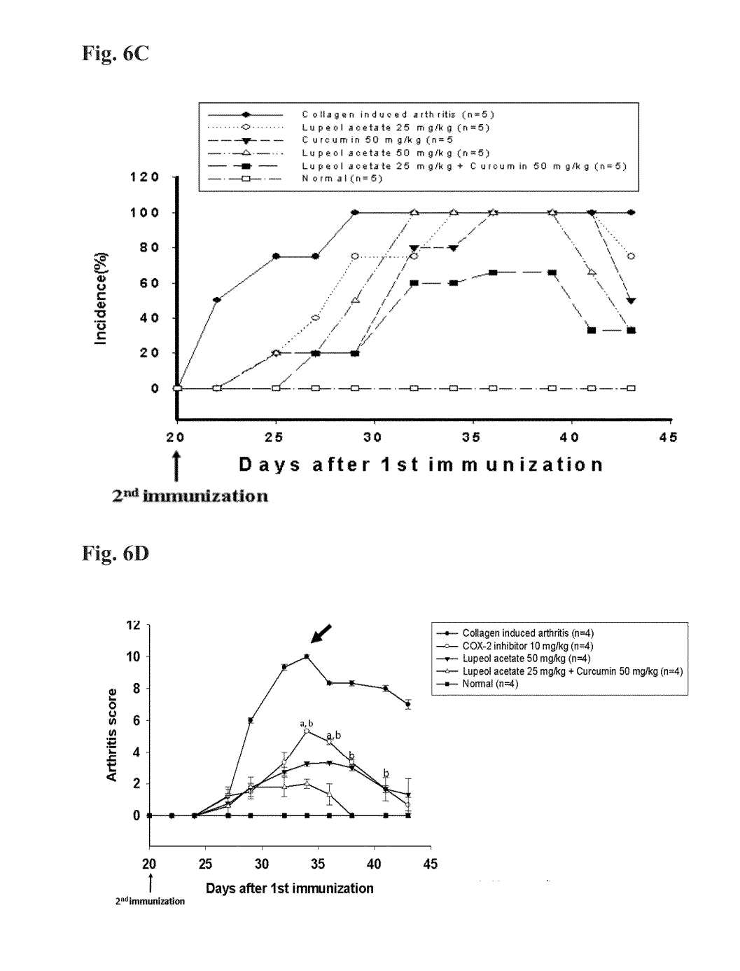

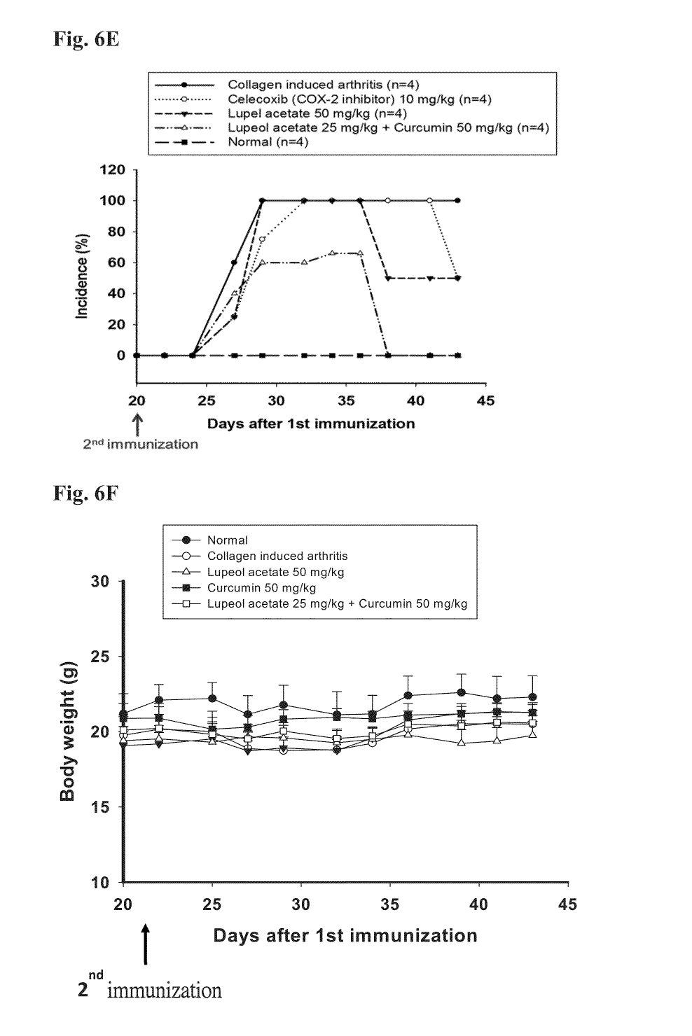

FIG. 6A shows that the arthritis onset of clinical symptoms in combination (LA+Cur) treated mice were significantly delayed as compared with non-treated CIA mice. From the results as shown in FIG. 6B and FIG. 6D, it is indicated that the severity of clinical score can be alleviated by the combination treatment, and there are significant differences in the therapeutic efficacy between the groups of LA, Cur or combination treated mice and arthritis mice. FIGS. 6C and 6E show the incidence of CIA in drug treated mice. The results indicate that combination group has significantly reduced the onset of collagen-induced arthritis. Through observed the change of body weight, we found that under the condition of these drug, there is no general toxic effect to the mice. Notably, the therapeutic effect in combination (25 mg/kg LA plus 50 mg/kg curcumin) treated mice group is better than that in Celecoxib group (treated with 10 mg/kg Celebrex®, a COX-2 inhibitor, for clinical treatment of RA).

The body weight tracking shows that changes were with ±10%, that means no general toxicity was found throughout the experimental period (FIG. 6F). It is indicated that LA, Cur and LA+Cur were well-tolerated. In the experiments above, the body weight of the mice has no significant change but the incident rate of arthritis has significantly reduced after combination treatment.

Apart from blood analysis, current clinical diagnosis will also use X-ray as an evaluation basis. Since X-ray image can only diagnose rheumatoid arthritis when the bone has been damaged severely at late stage, it could not be an effective evaluation tool. ¹⁸F-FDG can be used as a tracer to observe and evaluate its accumulation during rheumatoid arthritis. Therefore, in further experiments, we choose ¹⁸F-FDG to tract the inflammation in the joint of mice, and use micro-CT to prove the treatment group could diminish the bone loss.

Repeated ¹⁸F-FDG PET in vivo determination of disease severity in knee joints and paws on 20, 25, 32, 39 and 43 days after arthritis induction. Mice were anesthetized using 1-3% isoflurane and were intravenous injected with 0.5 mCi/100 μl (18.5 MBq) of ¹⁸F-FDG 40 min before images acquisition. The emission scans were performed using an animal micro/PET scan (Tri-Modality FLEX Triumph™ Pre-Clinical Imaging System). All images were acquired for 40 min and reconstructed by software supplied by the manufacturer. The ROIs were selected, and the accumulation of ¹⁸F-FDG of each ROI was further analyzed by Amide software.

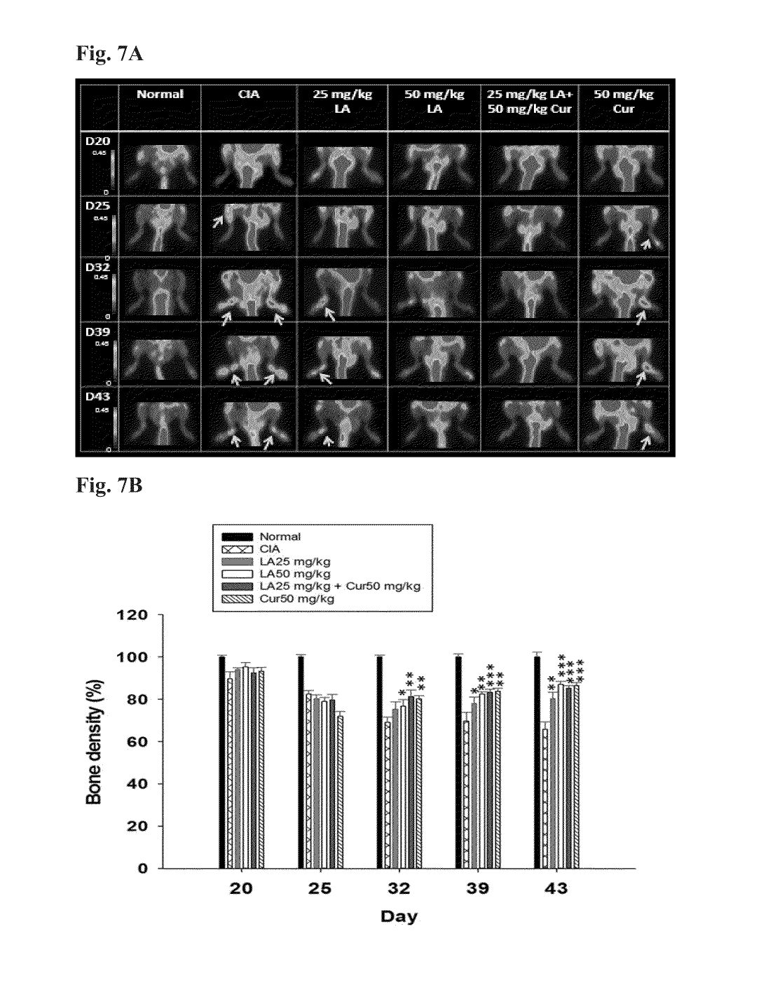

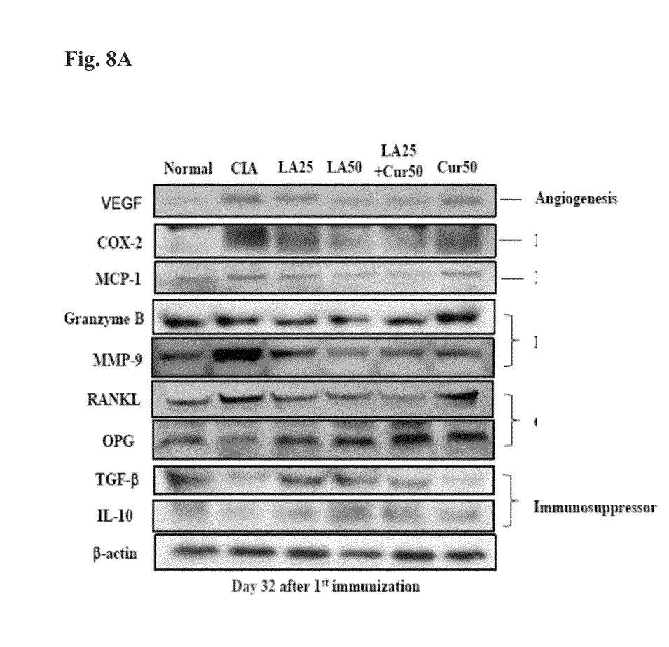

The results are shown in FIG. 7A. The accumulation of ¹⁸F-FDG in the knee and tarsal joint of CIA mice were increased up to day 32, and mice treated with LA 25 mg/kg alone, Cur 50 mg/kg alone still have leg swelling on one side. The combination-treated mice remained the same condition as compared with normal and LA 50 mg/kg treated mice.

Micro-CT images were acquired from the Tri-Modality FLEX Triumph™ Pre-Clinical Imaging System (Gamma MedicaIdeas; Northridge, Calif.). The ROI was analyzed visually in three different slices: transverse, coronal, and sagittal. Individual ROI was used to create 3D representations of the bones of interest. For each specific bone ROI, the total number of pixels and mean pixel intensity across all slices were used to measure bone volume and density. As shown in FIG. 7B, there was no significant difference between the initial bone densities of the treated mice on day 20 and 25. During subsequent scans on day 32, 39 and 43, significant differences were observed in drug treated mice when compared with CIA mice. The CIA mice showed a 34.1% decrease as compared with the normal group during the period of experiment.

### Example 6

### Combination of Lupeol Acetate and Curcumin Inhibits the Expression of RA-Related Proteins and Reduces the Activity of NF-κB

The mice were sacrificed by cervical dislocation on the 32^{nd} day of the animal experiment. The whole leg of mouse was removed, and the leg tissue was ground by adding an appropriate amount of lysis buffer (tissue protein extraction reagent, T-PER, Pierce Protein Biology Products, IL, USA), centrifuged at 15,000 rpm for 20 minutes, and the supernatant is taken as the sample for each group. The protein levels of angiogenesis, cell migration, bone erosion and immunosuppressor factor, including VEGF, COX-2, MCP-1, TGF-β, IL-10, Granzyme B, MMP-9, OPG and RANKL, were confirmed with ex vivo Western blotting.

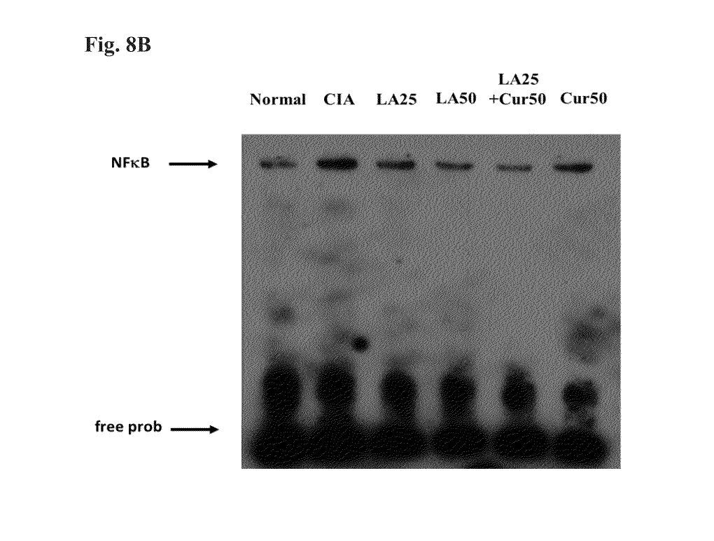

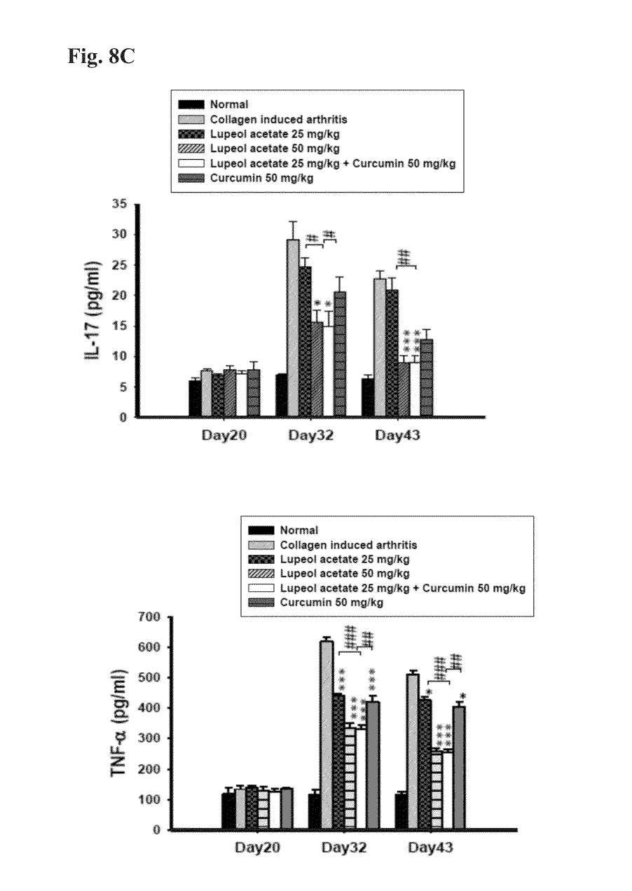

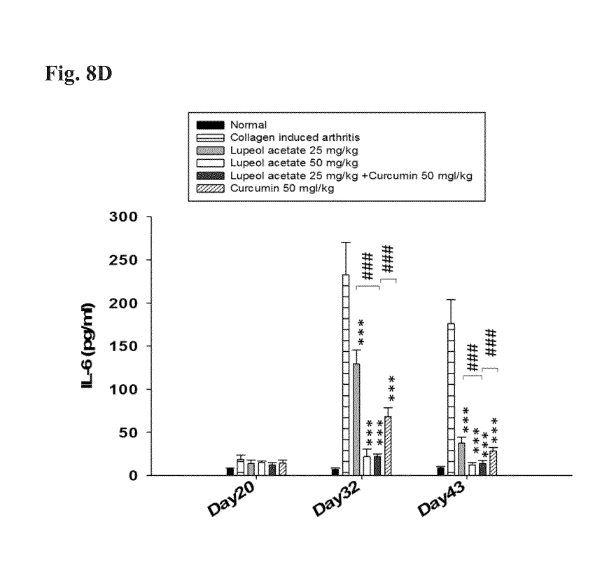

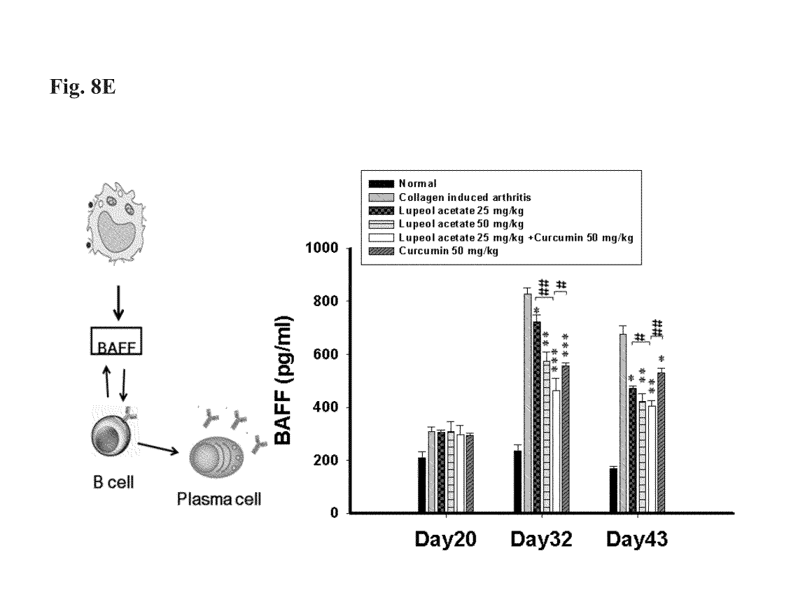

As shown in FIG. 8A, the angiogenesis and migration factor were decreased after combination treatment. Additionally, the immunosuppression of Treg cells could be expound through secretion of cytokine such as TGF-β and IL-10, and were both increased. Also, the bone damage related protein levels such as Granzyme B, MMP-9 and RANKL were all inhibited and the osteoclastogenesis inhibitory factor, OPG, was significantly increased in the combination group. It means that LA combined with curcumin could regulate the level of these inflammation and immunosuppression related proteins.

The LightShift Chemiluminescent EMSA kit (Pierce, Rockford, Ill., USA) was used in the analysis for NF-κB/DNA binding activity. Nuclear extracts were incubated with the biotin labeled DNA probes at room temperature for 20 minutes. The separated DNA/protein complexes from the free oligonucleotide on 10% polyacrylamide gel were transferred to a nylon membrane. The nylon membrane was immersed in ECL (Pierce, Rockford, Ill., USA) and reacted to emit cold light (luminescence), and then exposed to the film for the observation of NF-κB activity. Using IMAGE J software (National Institutes of Health), the obtained images were quantified to blackening degree; the blackening degree of the protein to be observed is divided by the value obtained in the control group to compare the differences of each group in the expression level of nuclear proteins. The result of ex vivo electrophoretic mobility shift assay (EMSA) shown in FIG. 8B indicates that the combination treatment exactly suppressed the activation of NF-κB.

FIGS. 8C-8E show the expression levels of IL-17, TNF-α, IL-6 and BAFF in the serum isolated from the mouse cheek blood sampled on day 20, 32 and 43 after the first immunization. As shown by the results, the expression levels of TNF-α, IL-17 (FIG. 8C) and IL-6 (FIG. 8D) and BAFF (FIG. 8E) reached the peak on day 32. The serum levels of these inflammatory cytokines are lowered in all drug treatment groups, and significantly decreased in the combination treated groups.

### Example 7

### Combination Treatment Promotes the Immunosuppressor Cells to Accumulate in Spleen and Lymph Node

Treg cell is an immunosuppression-related T cell, and usually less differentiated in autoimmune diseases. Some literatures have indicated that injection of Treg cells to the back of mice will effectively reduce the incidence of rheumatoid arthritis. Therefore, we implicate that the increase in the number of Treg cells induced by the treatment of the LA+Cur can reduce the occurrence of rheumatoid arthritis.

The mice were sacrificed at the peak of the incidence, that is the 32^{nd} day of the experiment. The spleen and drained lymph nodes (DLNs) were harvested and labeled with anti-FoxP3-Alexa Fluor 488/CD4-APC/CD25-PE Abs according to manufacturer's protocol of Mouse Treg Flow Kit (Biolegend, San Diego Calif., USA). The percentage of positive stained cells was analyzed by FACS instrument (BD Biosciences, San Jose, Calif., USA).

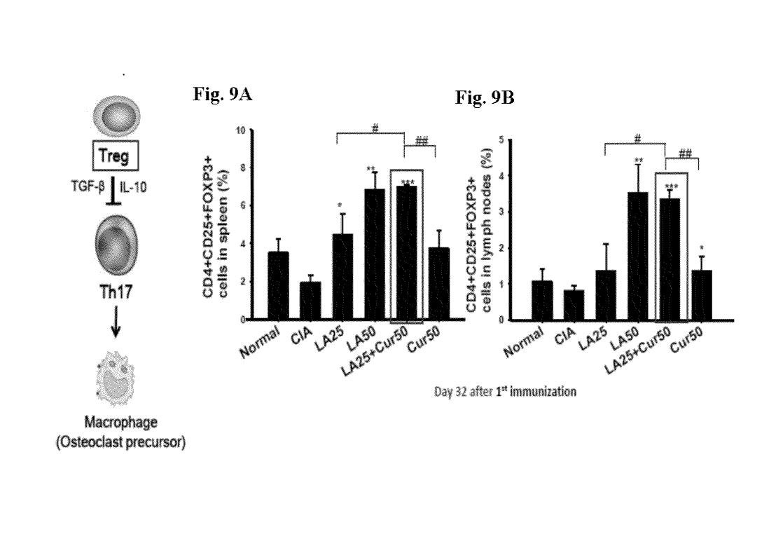

The results showed that the percentage of Treg cells in the combination group had no significant difference with that of the LA50 mg/kg group. Also, the combination group had a significantly higher percentage of Treg cells as compared to those of the other groups (FIG. 9A and FIG. 9B). These results are consistent with the previous results of mice serum with proinflammation cytokines, TNF-α, IL-17 and IL-6. Together, combination treated group has less inflammatory cell infiltration as compared with that of mice with RA. Both results are corresponding to each other.

### Example 8

### Comparison in the Therapeutic Efficacy of Combination (Lupeol Acetate Plus Curcumin) with Celecoxib (Celebrex® Capsule) in CIA Animal Model

Celecoxib (Celebrex® capsule) is a known COX-2 inhibitor, a kind of non-steroidal anti-inflammatory analgesics (referred to as NSAIDs), and currently used in the clinical treatment of menstrual pain or chronic pain caused by rheumatoid arthritis, degenerative arthritis. In this embodiment, the inhibitory effects of combination (lupeol acetate plus curcumin) on the expressions of arthritis-related proteins and osteoclastogenesis inducing factor RANKL are compared with the inhibition by Celecoxib. Furthermore, immunohistochemical staining was used to evaluate the inhibition of joint inflammation by these drugs.

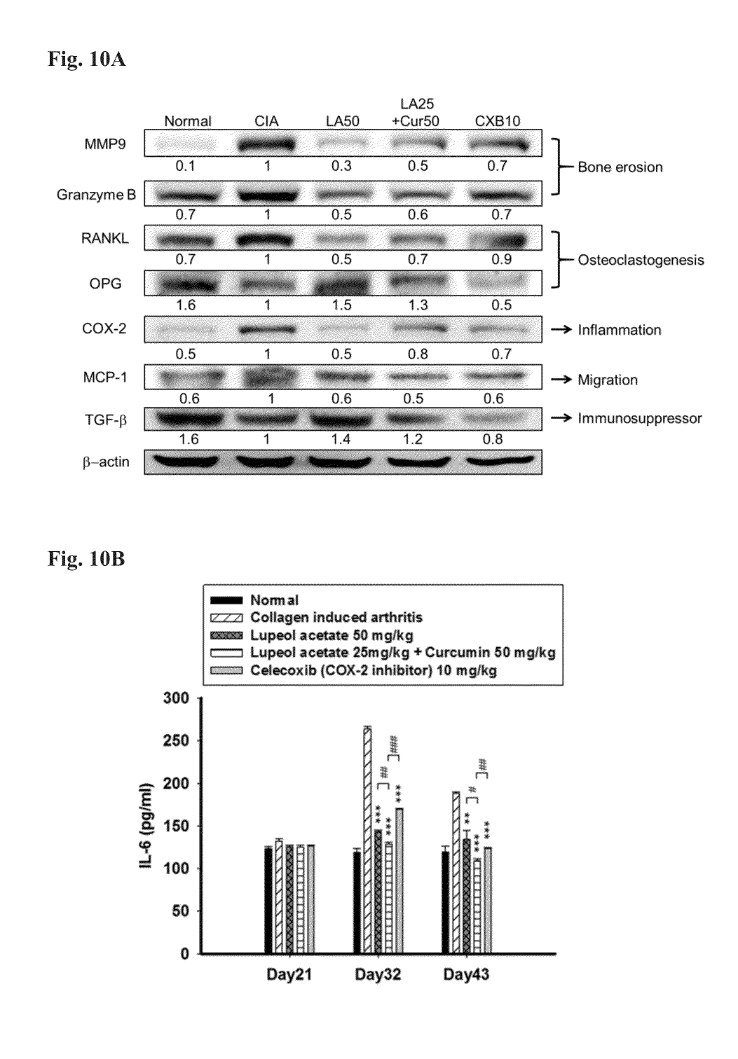

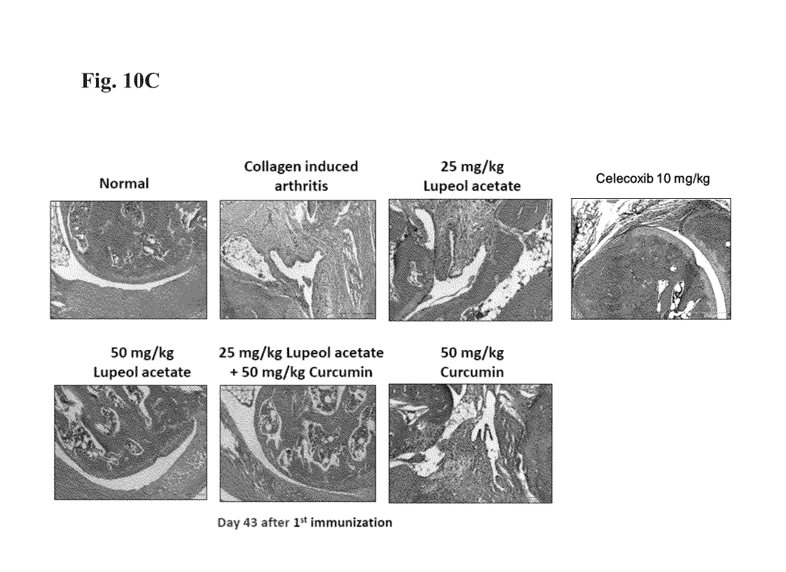

Mice were sacrificed at the peak of arthritis (on day 32), the protein extraction was isolated from legs of each group. The protein levels of angiogenesis, cell migration, bone erosion and immunosuppressor factor were confirmed with ex vivo Western blotting. As shown in FIG. 10A, the expression levels of cell migration-related (COX-2 and MCP-1) and bone erosion-related proteins (granzyme B and MMP-9), and the expressions of regulatory factors for osteoclastogenesis (RANKL) are decreased by the treatment of the combination more effectively than by the treatment of 10 mg/kg Celecoxib (CXB10). The expressions of immunosuppressive protein TGF-β and osteoclastogenesis inhibition factor OPG (Osteoprotegerin) are more significantly increased in combination treatment group than in Celecoxib treatment group and CIA group.

For the analysis of IL-6 expression, mouse cheek blood was sampled on day 20, 32 and 43 after the first immunization, and serum was isolated and subjected to ELISA IL-6 (Cat. No. 88-7064, eBioscience, CA, USA) according to the manufacturer's instruction manual. As shown in FIG. 10B, significant effect for reducing IL-6 level is observed in all drug treatment group, and combination (lupeol acetate plus curcumin) group produces more significantly inhibitory effect than Celecoxib (Celebrex®, Celebrex capsules) treatment.

To investigate the therapeutic efficacy of LA combined with curcumin on the histological damage in CIA mice, the mice were scarified on day 43 after arthritis induction. The joints of four limb of each mouse were excised and fixed with paraformaldehyde then embedded by paraffin and sectioned. Slides were stained with haematoxylin and eosin (H & E), and were observed under microscope.

As shown in FIG. 10C, CIA group showed both severe immune cell infiltration and bone damage as compared with those of the normal group. 25 mg/kg LA and 50 mg/kg curcumin treated mice showed moderate immune cell infiltration and cartilage destruction, while the combination treated mice and LA 50 mg/kg group had no inflammation and cartilage destruction similar to those of the normal mice, indicating the better therapeutic efficacy than other drug treatment groups, including 10 mg/kg Celecoxib group.

In summary of the experimental results described above, it has proven that combination of lupeol acetate with curcumin at half dosage can synergistically alleviate the inflammatory response by inhibiting the release of cytokines, such as COX-2, MCP-1, TNF-α, IL-1β and the like by macrophages, and reduce the expression of osteoclastogenesis-related proteins, such as MCP-1, COX-2, granzyme B, MMP-9, TGF-β, IL-1β, OPG and RANKL by regulating the levels of NF-κB and NFATc1. Moreover, it has demonstrated in the in vivo CIA animal experiments that combination of lupeol acetate plus curcumin of the present invention can effectively alleviate the joint inflammation, swelling, bone erosion and the incidence of rheumatoid arthritis in mice.

According to the present invention, the development of autoimmune arthritis is suppressed by the combination of lupeol acetate (LA) and curcumin (Cur) via blockading the release of pro-inflammatory cytokines and decreasing the formation of osteoclasts in vitro and in vivo. Also, the golden section of combination of LA+Cur (lupeol acetate 25 mg/kg plus curcumin 50 mg/kg) not only reduces the cost but also a potential treatment in patient with RA and osteoporosis. Accordingly, the present invention has reached the purpose in reducing the clinical dosage of lupeol acetate by combination with curcumin for the treatment of osteoclast precursor associated disorders, including rheumatoid arthritis (RA) or osteoclastogenesis-related diseases. The composition of lupeol acetate and curcumin at low dosage will be useful in inhibiting inflammation, improving the severity of bone erosion and joint swelling, and alleviating bone loss in arthritis patients.

In addition, the current therapy of rheumatoid arthritis is often in combination with steroid drugs, which will increase the chance of occurring side effects such as osteoporosis. Therefore, the present invention further provides a composition comprising lupeol acetate and curcumin combined with conventional steroids for the treatment of rheumatoid arthritis, which is beneficial to reduce the probability and severity of osteoporosis in the patients with rheumatoid arthritis by the effects on inhibiting osteoclastogenesis.

1. A composition for treating or preventing osteoclastogenesis associated disorder, comprising 25-50 mg/kg of lupeol acetate combined with 40-50 mg/kg of curcumin.

2. The composition of claim 1, further comprising a pharmaceutically acceptable vehicle, diluent or excipient,

3. The composition of claim 1, wherein the lupeol acetate and the curcumin are combined at a ratio of 0.5:1 to 1:2.

4. The composition of claim 3, wherein the combination ratio of lupeol acetate and the curcumin is in an range of 1:1 to 1:2

5. The composition of claim 1, which is used to inhibit the over-activation of macrophage.

6. The composition of claim 1, wherein the osteoclastogenesis associated disorder includes rheumatoid arthritis (RA).

7. The composition of claim 1, wherein the osteoclastogenesis associated disorder includes an osteoporosis.

8. The composition of claim 7, wherein the osteoporosis is a sterol anti-inflammatory agent triggered osteoporosis.
